(19) United States
(12) Patent Application Publication
(10) Pub. No.: US 2012/0245701 A1
(43) Pub. Date: Sep. 27, 2012

(54) Hemi Ankle Implant

(76) Inventors: Rudolf Zak, Voorhees, NJ (US); Yong Jae Kim, Voorhees, NJ (US)

(21) Appl. No.: 13/429,429

(22) Filed: Mar. 25, 2012

Related U.S. Application Data
(60) Provisional application No. 61/465,750, filed on Mar. 24, 2011.

(51) Int. Cl. A61F 2/42

(52) U.S. Cl. 623/21.18

(57) ABSTRACT

An ankle implant having a bearing, a tray and a bone screw. The tray is implanted to a talus bone. The tray includes a stem extending from the tray for connecting to the bone screw. The bone screw includes a shank and an enlarged head proximate its distal end. The bearing is connected to the tray.

### BACKGROUND OF THE INVENTION

The present invention relates to an orthopedic implant. In particular, the present invention relates to an orthopedic implant for an ankle joint.

Traditionally, treatment for ankle joint pain resulting from rheumatism, or degenerative or traumatic arthritis included either arthrodesis i.e., joint fusion, or total ankle arthroplasty. However, fusion of the ankle joint, renders the ankle stiff and generally immobile relative to the lower leg, resulting in limited use and additional stresses on the knee and hip joints, and adversely affecting gait. Moreover, to date, the success of total ankle arthroplasty has been met with only limited success, due in part to the complex motion/biomechanics of the ankle. As a result, there is still a need for an alternative to address ankle joint pain besides arthrodesis or total ankle arthroplasty. Such a need is addressed by the instant invention.

### BRIEF SUMMARY OF THE INVENTION

In a preferred embodiment, the present invention provides an orthopedic device that includes a curved body, a stem and a bone screw. The curved body is coupled to a resected talus bone and includes a curved superior surface, an anterior portion for positioning about an anterior of the resected talus bone, and a distal surface. The stem extends from the distal surface distally, posteriorly and laterally relative to the anterior portion. The bone screw is configured to connect with the stem.

In another preferred embodiment, the present invention provides an ankle implant that includes a tray, a stem, a bone screw and a bearing. The tray couples to a talus bone. The stem extends from the tray. The bone screw includes a shank having a proximal end configured to engage with the stem, and a distal end. The bone screw also includes an enlarged head proximate the distal end. The bearing is connected to the tray.

In yet another preferred embodiment, the present invention provides a method of implanting an ankle prosthesis that includes the steps of forming a through hole that extends through a talus bone and through a calcaneus bone, attaching a talar component to the talus bone, inserting a bone screw through a distal end of the through hole in the calcaneus bone, and connecting a proximal end of the bone screw to the talar component.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings an embodiments that is presently preferred. It should be understood, however, that the invention is not limited to the precise arrangements and instrumentalities shown.

In the drawings:

FIG. 1 is a side elevational view of a right ankle implated with an ankle implant in accordance with a preferred embodiment of the present invention;

FIG. 2 is a side elevational view of a bearing component and a talar plate of the ankle implant of FIG. 1;

FIG. 2A is a side elevational view of a bearing component and a talar plate in accordance with another aspect of the ankle implant of FIG. 1

FIG. 3 is an anterior elevational view of the bearing component and the talar plate of FIG. 2;

FIG. 4 is a side elevational view of a right talar plate in accordance with another preferred embodiment of the present invention;

FIG. 5 is a side elevational view of a right bearing component in accordance with another preferred embodiment of the present invention;

FIG. 6 is a perspective view of a bone screw of the ankle implant of FIG. 1;

FIG. 7 is a side elevational view of a bone screw for an ankle implant accordance with another preferred embodiment of the present invention; and

FIG. 8 is a perspective view a bone screw for an ankle implant accordance with yet another preferred embodiment of the present invention.

### DETAILED DESCRIPTION OF THE INVENTION

Certain terminology is used in the following description for convenience only and is not limiting. The words “right,” “left,” “upper,” and “lower” designate directions in the drawings to which reference is made. For purposes of convenience, “distal” is generally referred to as away from the center of the body, and “proximal” is generally referred to as closer to the center of the body. “Anterior” is generally referred to as the front of the body, “posterior” is generally referred to as rear of the body. Additionally, the term “a,” as used in the specification, means “at least one.” The terminology includes the words above specifically mentioned, derivatives thereof, and words of similar import.

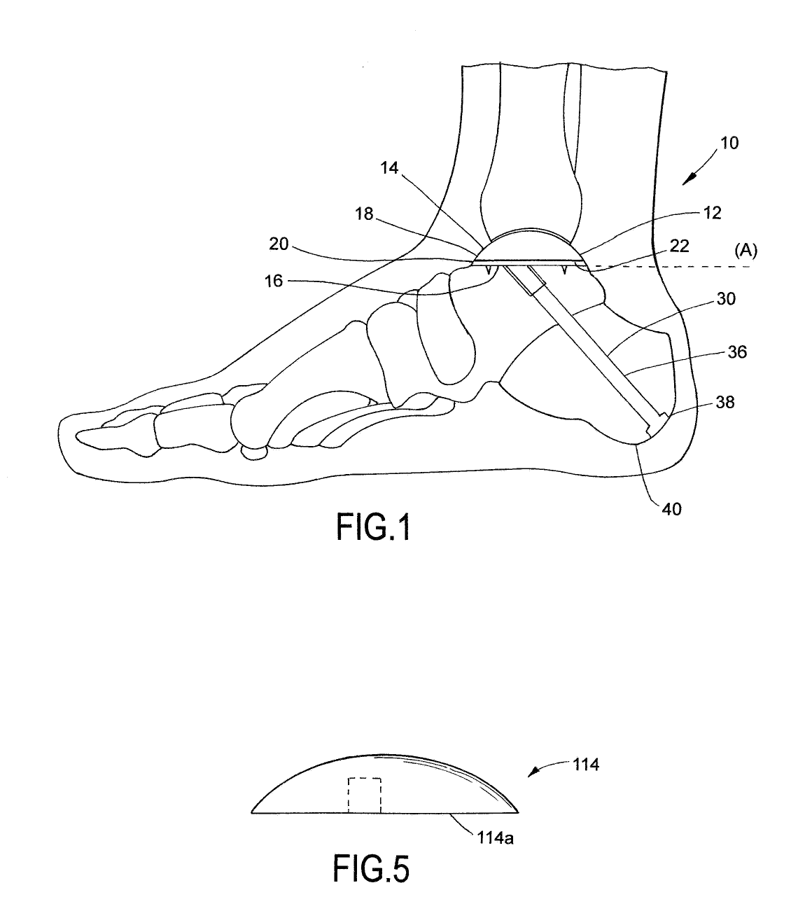

In accordance with a first preferred embodiment of the present invention, there is shown an orthopedic device 10 (also referred to as an ankle implant or ankle prothesis) that includes a talar component 12 and a bone screw 30, as shown in FIGS. 1-3 and 6 for a right-sided orthopedic device 10. For purposes of this embodiment, a right ankle implant is described for exemplary purposes. The orthopedic device 10 is a symmentrical device, such that the device 10 for a left-sided ankle is a minor image of a device for a right-sided ankle.

The talar component 12 includes a bearing 14 and a talar plate 16 securely fixated to the bearing 14, configured for coupling to a talus bone. The bearing 14 has a proximal end and distal end opposite the proximal end. The bearing 14 can be securely attached to the talar plate 16 by a mechanical means, such as a dovetail connection, screws, or with an adhesive, such as bone cement. Such means of fixedly attaching a bearing component to a plate are known in the art and a detailed description of such fixation means is not necessary for a complete understanding of the present invention. The talar plate 16 has an anterior portion 16a that is configured to be oriented in the anterior direction or substantially in the anterior direction when coupled to the resected talus bone.

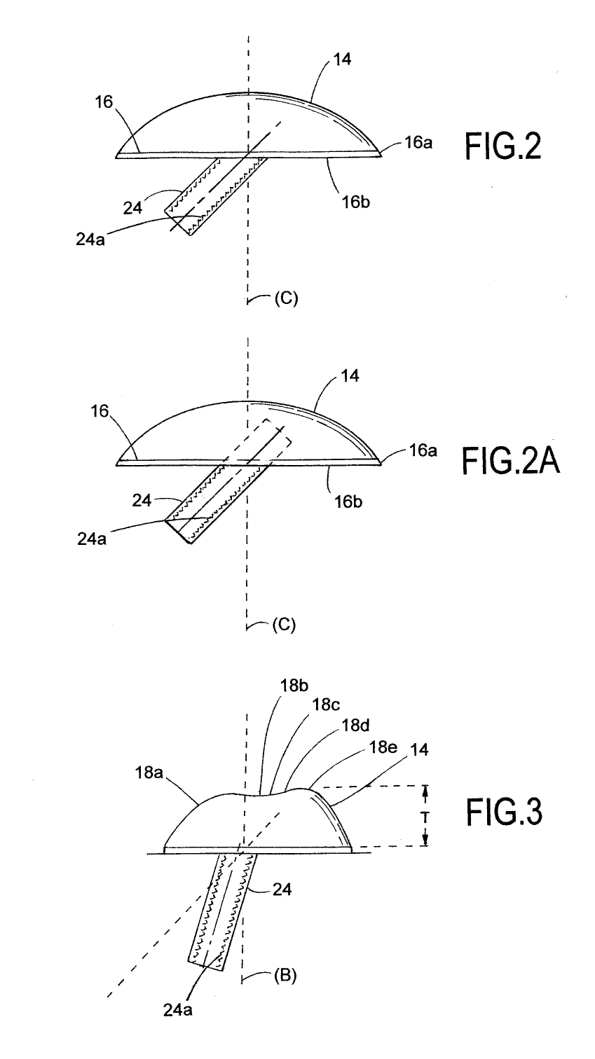

The bearing 14 is generally configured as a curved body that includes a curved superior surface 18 (i.e., a substantially proximally facing surface), an anterior portion 20 and a distal surface 22. The superior surface 18 is contoured to have a shape similar to i.e., mimicking the contours of a natural talus bone. Specifically, when viewed from an anterior view (FIG. 3) the bearing 14 has a cross sectional profile with a first arcuate portion 18a extending from the lateral side to a first point of inflection 18b. Extending from the first point of inflection 18b is a second arcuate portion 18c which extends to a second point of inflection 18d, positioned medially to the first point of inflection 18b. Extending from the second point of inflection 18d is a third arcuate portion 18e that forms the medial side of the bearing 14. The third arcuate portion 18e extends superiorly relative to the first arcuate portion 18a. When viewed from a lateral view, as shown in FIG. 2, the bearing 14 has a generally convex crossectional profile.

The bearing 14 has a thickness T (FIG. 3) of about 2, 4, 6, 8, 10, 12, 14, 16, 18 and 20 mm. Preferably, the bearing 14 for the orthopedic device 10 is provided with a variety of sizes to accommodate the natural variation in a patient's bone associated with human anatamony.

The bearing 14 can be made from any suitably strong and wear resistant material, such as, but not limited to polymers, including polyethylene and crosslinked polyethylene, a ceramic, a metal or combinations thereof. Preferably, the bearing 14 is formed from ultrahigh molecular weight polyethylene.

The talar plate 16 is generally configured, as shown in FIGS. 2 and 3. The talar plate 16 is configured to rigidly attach to the bearing 14. Specifically, the talar plate 16 is attached to the bearing 14 such that there is no movement between the talar plate 16 and bearing 14 or that movement of the bearing 14 relative to the talar plate 16 is minimized as much as possible. Preventing movement of the bearing 14 avoids or minimizes the frictional forces between surfaces of the bearing 14 and the talar plate 16, thereby avoiding or minimizing possible wear debris generated from such movement or motion.

Possible attachment mechanisms for connecting the bearing 14 to the talar plate 16, by way of example only and not by way of limitation, includes screws, a dove-tail connection, a snap-fit, and a snap-fit of the bearing into a talar plate having a retaining wall any other connection means suitable for its intended use.

Alternatively, the bearing 14 and talar plate 16 can be formed as a unitary structure. The unitary bearing 14 and talar plate 16 can be made from any suitably strong and wear resistant material, such as metal, plastics (including polymers) and the like.

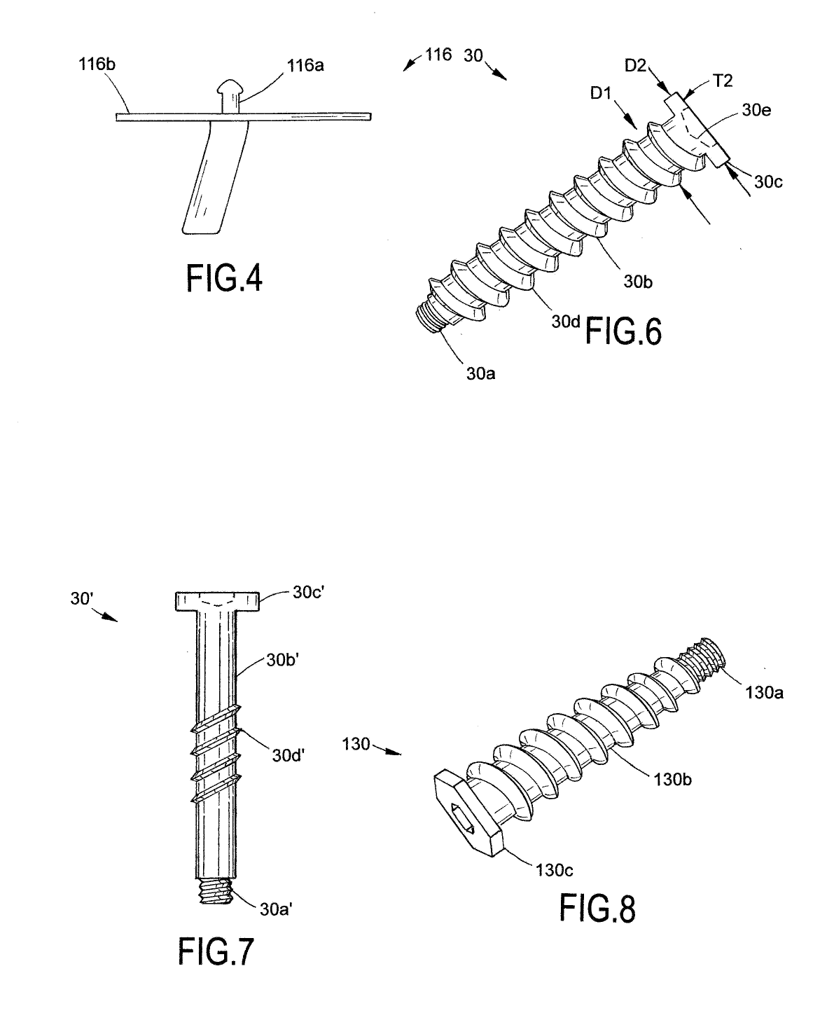

Alternatively the orthopedic device 10 can be configured as a mobile bearing device, as shown in FIGS. 4 and 5. That is, the bearing 114 and talar plate 116 can be configured as a mobile bearing device in which the bearing 114 is free to move relative to the talar plate 114 about an upper surface 116b of the talar plate. That is, the bearing 114 has a distally facing surface 114a that slidingly engages the upper surface 116b of the talar plate. In the mobile bearing configuration, the talar plate 114 is configured with a post 116a located subatantially centrally on the talar plate plateau 116b. The post 116a is configured to extend upward from the talar plate plateau 116b a distance from about ¼ to about ½ the height of the bearing 114. Further, the talar plate plateau 116b is configured to have a polish surface finish to reduce and minimize frictional forces of the talar plateau surface 116b, thereby minimizing any possible wear debris generation when in contact with the bearing 114.

The bearing 114 is configured with a cooperating female end or countersink 114a for receiving the post 116a. Additionally, the countersink 114a can be configured with an inwardly extending flange for engaging a proximal end of the post 116a for added fixation of the bearing 114 to the talar plate 116.

The overall peripherial profile of the talar plate 16, when viewed from a top plan perspective, is configured to substantially match the overall cross-sectional profile of a talar bone that has been traversed by plane (A), as shown in FIG. 1. Alternatively, the overall profile of the talar plate 16 can be configured into any suitable configuration that allows for a substantial portion of the resected talar bone to be covered by the talar plate 16. Preferably, the overall profile of the talar plate 16 is configured to cover or engage cortical bone of the talar bone.

Referring to FIG. 2, extending away from and at an angle from an inferior surface 16b (i.e., a bottom surface) of the talar plate 16 is a stem 24. Preferably, the inferior surface 16b is a planar surface. The stem 24 has a proximal end adjacent the inferior surface 16b and a distal end about an end of the stem opposite the proximal end. The stem 24 can be connected to the talar plate 16 by any suitable means readily known in the art. Preferably, the stem 24 is integrally fromed as a unitary structure with the talar plate 16. The stem 24 extends distally and laterally relative to the anterior portion 16a, as shown in FIG. 3, or relative to its attachment point to the talar plate 16 when the device 10 is coupled to a resected talus bone. In other words, the stem 24 is angled both from the planar inferior surface 16b and a saggital plane (B). Preferably, the stem 24 extends in the lateral direction from a saggital plane (B) an angle from about 65 to 80 degrees and more preferably from about 70 to 75 degrees.

The stem 24 also extends distally and posteriorly, as best shown in FIG. 2 relative to the anterior portion 16a or relative to its attachment point to the talar plate 16 when coupled to a resected talus bone. The stem 24 extends in the posterior direction relative to a coronal plance (C) an angle from about 35 to 50 degrees and more preferably from about 40 to 45 degrees.

The stem 24 includes female threads 24a about its distal end configured for connecting with male threads 30a of a bone screw 30 (FIG. 6), as further described below. The female threads 24a are preferably configured to extend into the stem 24 a partial distance of the total length of the stem 24.

However, the female threads 24a can alternatively be configured to extend the entire length of the stem 24. Additionally, when the female threads 24a are configured to extend the entire length of the stem 24, the bearing 14 can optionally be configured to include female threads configured to operate contiguously with the female threads 24a of the stem 24. That is, the bearing 14 is configured with female threads 24a′ oriented to line up with the threads 24 such that the bone screw 30 can threadly engage both the female threads 24a and the threads 24a′ in the bearing component 14 (see FIG. 2A)

Alternatively, the stem 24 and bone screw 30 can be configured with any other connection mechanism that allow for secure engagement of the screw 30 to the stem 24 at variable axial lengths along the stem's longitudinal axis.

The talar plate 16 can be made from any suitably strong material, such as, but not limited to titanium, cobolt chrome, a ceramic, and combinations thereof. Preferably, the talar plate 16 is formed from cobolt chrome.

The bone screw 30 is configured, as best shown in FIG. 6 and includes a shank 30b and a head 30c. The shank 30b has an overall maximum diameter of D1 and male threads 30d. Preferably the male threads 30d extend the entire length of the shank 30b, but can alternatively be configured to extend a partial length of the shank 30b, such as proximal end, a distal end, or a middle portion of the shank 30b. The bone screw 30 has a distal end (end proximate or near the head 30c) and a proximal end opposite the distal end. The proximal end is the end of the screw 30 that is opposite the end proximate or near the head 30c.

The head 30c is an enlarged or bulbous head, meaning that the head 30c has a diameter D2 that is larger than the overall maximum diameter of D1 and male threads 30d. Preferably, the head 30c has a diameter D2 that is 10%, 20%, 30%, 40% and 50% larger than D1. The head 30c also has an axial thickness T2, sufficient to give the head 30c structural support. The head 30c can optionally include a countersink or counterbore 30e for receiving a corresponding instrument for rotating or turning the bone screw 30, such as a hex driver, T-driver or a screw driver. In sum, the head 30c is configured as a radially outwardly extending flange that extends radially outwardly from an outer lateral surface of the shank 30b.

The bone screw 30 is preferably configured, as shown in FIG. 6, but can alternatively be configured as shown in FIG. 7. Referring to FIG. 7, the bone screw 30′ includes a proximal end having male threads 30a′ and a distal end having a head 30c′. The bone screw 30′ differs from the bone screw 30, in that the threads 30d′ are configured to extend only a partial length of the overall length of the shank 30b′. The threads 30d′ can be positioned about a distal region, a proximal region or a mid region of the shank 30b′. In one aspect of the present embodiment, the bone screw 30′ can be configured with a proximal end having threads 30a′ and shank region having threads 30d′ only about its mid portion, or threads 30d′ that is spaced from the threads 30a′ about the bone screw' proximal end.

The bone screw can alternatively be configured, as shown in FIG. 8, as a tapered bone screw 130. That is, the bone screw 130 has a shank 130b that is tapered. The taper can be in the proximal direction, such that the proximal end of the bone screw 130 has a smaller diameter than a diameter of a distal end of the bone screw 130. The distal end being closer to the head 130c. Alternatively, the shank 130b of the bone screw 130 can be tapered in the distal direction. That is, the shank 130b tapers inwardly going from a proximal end (proximate the threads 130a) to the distal end (proximate the head 130c).

Additionally, the bone screw 30 can be configured with threads having a variable pitch (not shown) or a variable pitch in combination with a tapered shank (not shown).

The bone screw 30 can be made from any suitably strong material, such as, but not limited to titanium, cobolt chrome, a ceramic, combinations thereof and the like. Preferably, the bone screw 30 is formed from cobolt chrome.

The orthopedic device 10 is also referred to as a hemi ankle implant 10, because unlike traditional total ankle implants, the orthopedic device 10 does not include a corresponding tibial component configured to articulate with the bearing 14. Instead the hemi ankle implant 10 consists essentially of the talar component 12 and the bone screw 30 and is configured to articulate with the natural bone of the tibia.

A hemi ankle implant 10 would provide a beneficial option to those patient's in which the tibia is not as effected, damages, or degraded as much as the talus. This option of a hemi ankle implant also preserves the tibial bone and minimizes natural bone loss associated with the talus. Thus, in the event a revision surgery is necessary, which is common for total ankle joint replacement and arthrodesis, the patient's bone stock will be preserved sufficiently, for example, a total ankle joint replacement and arthrodesis. That is, the hemi ankle implant 10 provides patients with a treatment option before the need to consider a more severe option, such as a total ankle joint replacement or arthrodesis procedure.

The hemi ankle implant 10 is designed to be a resurfacing implant in which the proximal talus is resurfaced, thereby minimizing bone loss. The hemi ankle implant 10 also fuses the talus and calcaneous bones together with the bone screw 30, thereby permanently joining and stabilizing the talus and calcaneous bones together. The reason for the fusion is to eliminate stresses on the ankle implant and provide stability. Motion across the subtalar joint transfers stresses directly to the ankle implant. That is, during normal gait, when the calcaneus everts the talus adducts and plantar flexes within the ankle joint. The combination of the resurfacing of the patient's talus with a fusion of the talus and calcaneous, not only provides for relief of pain associated with articulation of the ankle joint, but also better preserves the ankle's natural range of motion in all planes. As a result, a patient with the hemi ankle implant 10 will not suffer from the traditional complications of stiffness, stresses to hip and knee joints or gait implications associated with total ankle replacements and arthrodesis. In other words, the hemi ankle implant 10 provides for a minimally invasive surgical option for the treatment of e.g., but not limited to, rheumatism and degenerative or traumatic arthritis.

The hemi ankle implant 10 is implanted into a patient by resecting or resurfacing the proximal talus a depth equivalent to the overall thickness of the bearing 14 and talar plate 16, excluding the stem 24. Preferably, the talus is resected substantially horizontally relative to an axial anatomical plane or the ankle in flexion (i.e., foot approximately 90 degrees relative to the tibia). A through hole 36 is then formed starting at the resected proximal talus that extends in the direction of the stem 24. That is, the through hole 36 is formed to extend posteriorly and laterally through the talus and calcaneous. The orientation of the through hole 36 relative to the resected proximal talus substantially alignes with the orientation of the stem 24 of the talar plate 16 when oriented in the implanting position. The through hole 36 is sized in diameter to sufficiently received and engage the bone screw 30. A counterbore or recess 38 is formed on the distal end of the calcaneous proximate the through hole 36 for receiving the head 30c of the bone screw 30. The position of the counterbore/recess 38 and ultimately the final position of the head 30c of the bone screw 30 is sufficiently posterior of the heel 40 of the foot such that during normal gait, the head 30c of the bone screw 30 will not make direct contact with the ground surface.

In order to fuse the subtalar joint another incision will need to be made along the lateral aspect of the subtalar. The articular surface off of the posterior facet of the talus as well as the adjacent undersurface off the calcaneus will then need to be sufficiently resected.

The talar plate 16 is then attached to the resected talus with the anterior portion 16a of the talar plate 16 oriented anteriorly and the stem 24 inserted in the through hole. The talar plate 16 can be implanted either with the application of bone cement or press-fitted without any bone cement. For press-fitted applications, the talar plate 16 can be configured with an undersurface having a certain degree of porosity and/or coated with a hydroxyapatite based coating to promote bone growth, such as hydroxyapatite or Periapatite®.

The bone screw 30 is then inserted into the through hole via a distal approach. This is accomplished by inserting the proximal end of the bone screw 30 through the distal opening of the through hole. The male threads 30a is then extended through the through hole 36 sufficiently to engage the stem 24. The bone screw 30 is then connected with the stem 24 by threaded engagement of the corresponding threads of the bone screw 30 and the stem 24. The threaded engagement of the bone screw 30 with the stem 24 advantageouly provides compression of the talus and calcaneus which therery results in fusion of the subtalar joint. When fully assembled, the head 30c of the bone screw 30 is positioned within the counterbore 38 formed in the proximal calcaneous.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An orthopedic device comprising:
a curved body for coupling to a resected talus bone, the curved body includes:
a curved superior surface,
an anterior portion for positioning about an anterior of the resected talus bone, and
a distal surface,
a stem extending from the distal surface distally, posteriorly and laterally relative to the anterior portion; and
a bone screw configured to engage with the stem.

2. The orthopedic device of claim 1, wherein the bone screw has a proximal end and a distal end opposite the proximal end, the bone screw including:
a shank; and
an enlarged head proximate the distal end.

3. The orthopedic device of claim 2, wherein the stem includes female threads about a distal end of the stem, and wherein the bone screw includes male threads about its proximal end that engages the female threads of the stem.

4. The orthopedic device of claim 2, wherein the shank is a tapered shank that tapers inwardly and proximally.

5. The orthopedic device of claim 4, wherein the shank includes threads having a variable pitch.

6. The orthopedic device of claim 2, wherein the enlarged head is tapered.

7. The orthopedic device of claim 2, wherein the enlarged head has an overall diameter larger than an overall diameter of the shank.

8. The orthopedic device of claim 2, wherein the curved body has a planar distal surface and the stem extends in a posterior direction relative to the planar distal surface from about 35 to 50 degrees.

9. The orthopedic device of claim 2, wherein the curved body has a planar distal surface and the stem extends in a posterior direction relative to the planar distal surface from about 40 to 45 degrees.

10. The orthopedic device of claim 2, wherein the curved body has a planar distal surface and the stem extends in a lateral direction relative to the planar distal surface from about 65 to 80 degrees.

11. The orthopedic device of claim 2, wherein the curved body has a planar distal surface and the stem extends in a lateral direction relative to the planar distal surface from about 70 to 75 degrees.

12. An ankle implant comprising:
a tray for coupling to a resected talus bone;
a stem extending from the tray;
a bone screw that includes:
a shank having:
a proximal end configured to connect to the stem, and
a distal end, and
an enlarged head proximate the distal end; and
a bearing connected to the tray.

13. The ankle implant of claim 12, wherein the enlarged head has an overall width larger than an overall width of the shank.

14. The ankle implant of claim 12, wherein the shank includes threads about its proximal end for threadly engaging the stem.

15. The ankle implant of claim 12, wherein the tray includes a superior surface, an anterior portion for positioning about an anterior of the resected talus bone, and a distal surface; and wherein the stem extends from the distal surface of the tray distally, posteriorly and laterally relative to the anterior portion.

16. The ankle implant of claim 12, wherein the bearing includes:
an articulating surface;
a distal surface in facing engagement with a superior surface of the tray; and
a recess configured to fixedly engage the proximal end of the bone screw.

17. The ankle implant of claim 12, wherein the bone screw threadly engages and passes through the stem.

18. The ankle implant of claim 12, wherein the tray has a planar distal surface for coupling to the resected talus bone and the stem extends in a posterior direction relative to the planar distal surface from about 35 to 50 degrees.

19. The ankle implant of claim 12, wherein the tray has a planar distal surface for coupling to the resected talus bone and the stem extends in a lateral direction relative to the planar distal surface from about 65 to 80 degrees.

20. A method of implanting an ankle prothesis comprising:
forming a through hole that extends through a talus bone and through a calcaneus bone;
attaching a talar component to the talus bone;
inserting a bone screw through a distal end of the through hole in the calcaneus bone; and
connecting a proximal end of the bone screw to the talar component.
